(19) United States
(12) Patent Application Publication
(10) Pub. No.: US 2022/0032033 A1
(43) Pub. Date: Feb. 3, 2022

(54) IMPROVED INTRA-AORTIC BALLOON PUMP

(71) Applicant: Memory Metal Holland BV, Enschede (NL)

(72) Inventors: Petrus A. Besselink, Enschede (NL); Gerhard Rakhorst, Enschede (NL); Massimo Mariani, Enschede (NL); Michiel Elardus Erasmus, Enschede (NL)

(73) Assignee: Memory Metal Holland BV, Enschede (NL)

(21) Appl. No.: 17/275,257

(22) PCT Filed: Sep. 13, 2019
(86) PCT No.: PCT/IB2019/001034

§ 371 Date: Mar. 11, 2021

Related U.S. Application Data
(60) Provisional application No. 62/731,321, filed on Sep. 14, 2018.

(51) Int. Cl. A61M 60/139; A61M 60/497

(52) U.S. Cl. CPC A61M 60/139; A61M 60/497

(57) ABSTRACT

An intra-aortic balloon pumping device and a method of assembling an intra-aortic balloon pumping device. The device includes a catheter with a separated first and second lumen for driving a first balloon with a relatively large outer diameter and a second balloon with a smaller outer diameter than the aorta when inflated, as well as a single driver unit that is coupled to the first and second lumen for pumping a driving gas into and out from each individual lumen to inflate and deflate the first and second balloons in sequence. The ratio of cross-sectional area of each lumen and the balloon volumes are dimensioned in such way that the sequence is optimized. In one form, the second lumen is a short aperture, located only between adjacent chambers that are formed by the first and second balloons.

### CROSS-REFERENCE TO RELATED APPLICATIONS

This application claims the benefit of U.S. Provisional Application Ser. No. 62/731,321, filed Sep. 14, 2018.

### BACKGROUND

This disclosure relates to the field of producing an improved Intra-Aortic Balloon Pump (IABP), which can be used to support the heart function.

This disclosure relates generally to temporary cardiac assist devices used to assist the operation of a failing, traumatized or infarcted heart for a limited period of time until either the heart, recovers or a more definitive treatment can be provided. In particular, it relates to the so-called Intra-Aortic Balloon Pumps. Such a pump does not require major thoracic surgery to connect it to the circulation. When the balloon is maximally deflated, it can easily be introduced through a transcutaneal catheter into the femoral artery and may then be guided into some portion of the aorta, usually the thoracic aorta, where it can be employed to assist the left ventricle of the heart, that pumps the blood into the systemic circulation. When this left ventricle fails it might need mechanical support. In the usual “counter pulsation” mode of employment, the IABP balloon is pneumatically inflated during diastole to increase blood pressure and deflated during systole to lower the afterload of the left ventricle. This device and its mode of operation was described in a paper by Moulopolous, Topaz and Kolff, “Diastolic Balloon Pumping in the Aorta—A Mechanical Assistance to the Failing Circulation”, American Heart Journal (1962) 63, p. 669.

The current IABP, in which a single balloon inflates and deflates, works bi-directional, that is, it pushes some of the blood forward, perfusing to the lower body, and also pushes some of the blood towards the aortic root. This has been considered as increasing blood flow into the arteries branching off the aortic root, i.e., to the coronary arteries. However, this device can cause other problems, especially when the left ventricle severely fails, as indicated below.

In the literature, many solutions have been suggested to improve the behavior of IABP systems. Exemplary conventional cardiac assist systems of this type are disclosed in U.S. Pat. Nos. 4,080,958, 4,692,148, 4,077,394, 4,154,227 4,522,195, 4,407,271 and 4,697,574, the disclosures of which are hereby incorporated by reference herein in their entirety.

Conventional IABP's belong to the category of Left Ventricular Assist Devices (LVADs) or Cardiac Assist Devices. The balloon is placed in the aorta and stay by means of a catheter tube in connection with a driver unit that is located outside of the patient's body. The driver is ECG controlled in such a way that it can inflate and deflate the balloon with a frequency that is related to the heart-beating rate. The timing and duration of the inflation is very critical, because the aortic valve is closed only for a short time starting at the end of the contraction of the left ventricle and during the diastole. During this period, the created counter pulse will improve the flow of blood into the coronary arteries. Due to this bi-directional pulse generation, the forward pumping function of such a single IABP is not very efficient as the flow is not directed and furthermore much off the energy is absorbed by elasticity of the vessel wall. In principle, the blood flow caused by the balloon is bi-directional. When one or two one-way valves are combined with such a balloon that is placed in the thoracic aorta, the combination will work as an active pump that helps to increase the total blood flow by directing the blood flow distally, so it becomes more unidirectional.

In U.S. Pat No. 6,210,318 a description is given for U.S. Pat. Nos. 5,820,542 and 5,827,171, which disclose various complex designs for intravascular circulatory assist devices involving a pumping membrane such as an inflatable balloon, disposed within an expandable housing structure such as another balloon. The pumping membrane thus divides the outer housing into an intermediate control chamber and an interior pumping chamber. Injection and evacuation of a control/fluid into the control chamber deflates (pumps) and inflates (refills) the pumping chamber. Expandable and collapsible stents are disclosed as one mechanism to expand and retain the control chamber in its maximum dimension while control fluid is withdrawn.

U.S. Pat. Nos. 4,902,272 and 4,785,795 represent important advances in the art of cardiac support systems. Unlike the above cardiac assist systems that adjust systemic pressure to assist a natural heart, these latter patents disclose apparatuses and techniques for directly pumping blood. U.S. Pat. No. 4,902,272 discloses a catheter-based intra-arterial cardiac support system that includes one or two valves that are mounted upstream and downstream of a cyclically inflatable pumping balloon synchronized with the cardiac cycle. One disclosed embodiment provides assistance to the left ventricle through the placement of the pumping balloon in the descending aorta with a balloon valve located distally relative to the natural heart. The balloons are individually inflated and deflated, timed in order to directly pump blood. The pumping action is peristaltic in nature and operated in phased relationship to the systole and diastole of the natural heart. However, each balloon needs a separate inflation and deflation means to create the correct timing, thus making the system rather complicated.

U.S. Pat. No. 4,785,795 discloses a catheter-based, high-frequency intra-arterial cardiac support system that includes an externally controlled pumping balloon and balloon valve. The pumping balloon and valve are positioned in a major artery downstream of the natural heart and are operated at a pumping frequency that is at least three times the normal frequency of the heart to directly pump blood. To assist a left ventricle, for example, the balloon pump is located in the ascending aorta between the aortic valve and the ostium innominate artery. The pumping balloon and valve are sequentially operated to pump blood from the left ventricle into the arterial tree. To assist the right ventricle, the pumping balloon is located in the pulmonary track immediately downstream from the pulmonary valve. The pumping balloon and valve are sequentially operated to pump blood from the right ventricle into the pulmonary trunk. In each application, the balloon valve is positioned downstream of the pumping balloon; that is, the pumping balloon is positioned between the balloon valve and the natural aortic or pulmonary valve.

Although these approaches overcome the above-noted drawbacks associated with traditional cardiac assist systems by directly pumping blood to support or replace the pumping action of the heart, they also have limits to their effectiveness. Unlike conventional pumping balloons, the latter two approaches operate with the pumping balloon interposed between two valves in an otherwise closed region of the circulatory system. The valves may be natural or balloon valves, depending on the embodiment of the cardiac support system. U.S. Pat. No. 6,210,318 disclosed that at times during certain operations of such devices, the surrounding valves simultaneously occlude the vessel at least momentarily while the pumping balloon deflates. Such an occurrence creates temporarily a vacuum within the vessel region. At times this vacuum is sufficient to draw the vessel walls inward with the deflating pumping balloon. This reduces the effective pumping displacement of the pumping balloon, thereby reducing the overall effectiveness of these cardiac support systems.

In PCT Published Application No. WO 2018/158,635 a combination is disclosed of an IABP system with one or two expandable and collapsible one-way valves with expandable nitinol frames, that cause a unidirectional flow in the descending aorta upon inflation of the balloon. In another embodiment of this application, the normally passive one-way valve can be made inflatable by using double leaflets, which may be closed by raising the pressure between the valve leaflets. This leads to making it a remote-controlled occlusion valve that may be used to occlude the aorta in specific moments of the pumping cycle to improve the overall performance by replacing the normally passive opening and closing in response to changes in the blood pressure with inflation and deflation achieved independently from the blood flow. The combination of the valve with the balloon pump ensures that the timing of the remote closure of this valve opens new ways to ensure a better perfusion in the coronary arteries in the diastolic phase of the heart beating sequence, amongst others. In addition, while the valve is supposed to reach the inner wall of the aorta to ensure full closure, the longer balloon of the pumping section does not have to fully expand until it reaches the wall as the pumping is achieved by the volume change of the IABP balloon. Nevertheless, this system may cause damage to the inner wall of the aorta, because of minor movements of the valve frame metal along the wall.

U.S. Pat. No. 6,468,200 discloses a segmented peristaltic intra-aortic balloon pump, positioned on the distal portion of the catheter and which has three or more chambers in succession. In a preferred embodiment, first, second, and third chambers are arranged from distal to proximal on the catheter, and there are apertures formed in the catheter for communicating driving gas between the lumen and the chambers, respectively. This permits gas in the lumen to inflate and deflate the first, second and third chambers in such a way that the chambers inflate in sequence from distal to proximal and then deflate in sequence from distal to proximal. A pumping device outside the patient's body is connected to the lumen at a proximal end of the catheter. In a preferred mode, the three (or more) chambers are successively larger in the direction from distal to proximal, i.e., with the smallest chamber being closest to the aortic root Also, the apertures or openings from the lumen to the chambers are largest for the most distal, i.e., first, chamber and then progressively smaller for the second chamber, third chamber, and so on. This arrangement ensures that the first chamber will inflate first, then the second, and then the third, which causes peristaltic pumping toward the lower arteries. Similarly, the first chamber will deflate first, followed by the second chamber, and then the third chamber. This creates a negative pressure just prior to systole, to alleviate end-diastolic pressure on the left ventricle and relieves the afterload of the left ventricle. This also avoids flow towards the aortic root, which can cause problems if the patient's heart has aortic regurgitation. The problem with this segmented system is that all balloons are connected to the driver unit via the same lumen. Only when all balloons are filled completely, the pressure can rise. In intermediate stages of inflation, it would not be possible to reach a full occlusion of the aorta by the balloon that is located closest to the aortic root.

### SUMMARY

Accordingly, it is an object of the disclosure to provide a simple, reliable system, based on the combination of at least one relatively long repeatedly inflatable and deflatable balloon with a diameter smaller than the aorta, but with a large volume, with a smaller volume short balloon that reaches its full loading pressure much faster than and independently from the long balloon, while both balloons are connected to the same driver. The smaller volume short balloon almost or entirely occludes the aorta while the long balloon just started to inflate, and the long balloon reaches its largest diameter while the short balloon is under full pressure.

Preferably the system according to this disclosure can be used with standard drivers for single IABP devices without needing any major change of the existing drivers.

The disclosed configuration gives an optimization of the coronary perfusion during diastole by the rapid counter-pulsation, caused by the smaller volume short balloon. It further gives a better downward unidirectional flow into the descending aorta. Upon deflation, the small balloon deflates a lot faster than the long balloon, which causes a desirable unloading of the left ventricle. Other features caused by adding the short occlusion balloon include limiting whipping by the large balloon, keeping the large balloon in the center of the aorta, maintaining the axial position of the catheter, limiting energy losses and enabling the use of smaller balloons for requiring the same downward flow.

According to an aspect of the present disclosure, an intra-aortic balloon pumping device is disclosed. According to another aspect of the present disclosure, a method of assembling an intra-aortic balloon pumping device is disclosed.

### BRIEF DESCRIPTION OF THE DRAWINGS

The following detailed description of the preferred embodiments of the present disclosure can be best understood when read in conjunction with the following drawings, in which the various components of the drawings are not necessarily illustrated to scale:

FIG. 1 shows a schematic cross section of the aorta with therein an IABP catheter with a short occlusion balloon plus a long intra-aortic balloon pump;

FIG. 2 shows a detailed cross section of section A-A of the catheter of FIG. 1, showing three lumens, wherein two lumens are used for the medium (e.g. helium), and the third lumen is a guide wire lumen;

FIG. 3 shows a schematic cross section of the aorta plus another embodiment according to the disclosure where a small restriction is built in, with one or more small holes that allow the medium to flow from the inside of the short balloon into the inside of the long balloon; and

FIG. 4 shows a detailed cross section of section B-B of the catheter of FIG. 3, showing only two lumens, one used for the medium (e.g. helium), and the second lumen is a guide wire lumen.

### DETAILED DESCRIPTION

The advantages of the disclosed device and method will become more apparent after reference to the following description, wherein some embodiments are elucidated.

There are several options to make a combination of a small occlusion balloon with a large non-occluding balloon according to the present disclosure, and the embodiment that is described hereafter is only meant to show the principle.

In embodiments of the present disclosure, either the inflatable valve mentioned previously in conjunction with PCT Published Application No. WO/158,635, or a small short occlusion balloon, can be combined with a balloon pump according to this disclosure.

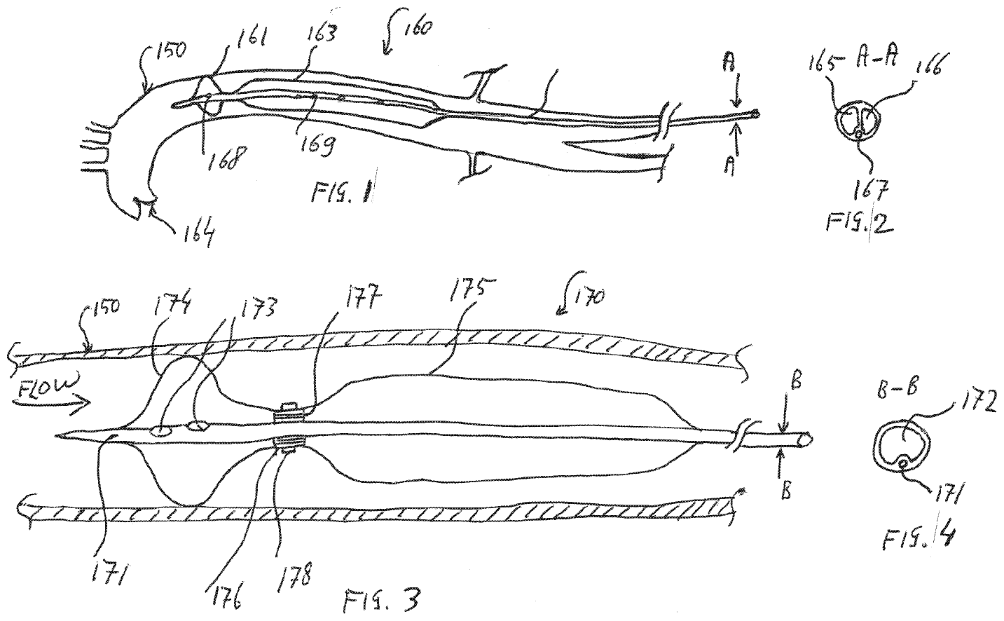

FIG. 1 gives a schematic cross section of the aorta 150 with therein an example of such a device 160, with a short first balloon (also referred to herein as a short small volume first occlusion balloon, short first occlusion balloon, first occlusion balloon, short balloon or occlusion balloon) 161 that is mounted at the distal end of a catheter 162 which also carries a long intra-aortic balloon (also referred to herein as long balloon) 163 that acts as a pump. The first occlusion balloon 161 has a small volume, but a large diameter when inflated, large enough to reach full or almost full occlusion of the aorta. The second balloon 163 is a lot longer than the first occlusion balloon 161, but it has a smaller diameter. The first occlusion balloon 161 is placed closest to the heart valve 164, so the blood flow is from left to right in this figure. Within the present disclosure, it will be understood that while normally the side of the heart is defined as the most proximal side and the extremities being distal, the distal end of the catheter is defined as the end that is located closest to the heart valve, while the end that is connected to the driver unit is defined as proximal. As such, these terms when used in the claims are understood to describe the embodiments of the device 160 and not the arterial system.

Here the cross-section A-A of catheter 162, as shown in detail in FIG. 2 needs at least three lumens, the lumens 165 and 166 for the medium (e.g. helium), plus a guide wire lumen 167. Lumen 165 is in open contact with the first occlusion balloon 161 via opening 168. Lumen 166 is in open contact with the second balloon 163 via openings 169. The size of openings 168 and 169 is sufficient to ensure an unobstructed flow of the medium into and from the respective balloons. Optionally there may be another lumen besides the guidewire lumen 167, for example for pressure sensing at the catheter tip.

By pumping helium or another type of medium into the catheter lumen 165 shown in cross section in FIG. 2, the short balloon can entirely close the aorta when it is inflated far enough. The correct timing of this closure is controlled via a driver unit that monitors the heartbeat, and that also causes the deflation of this short balloon in the right moment. Inflation and deflation of the long balloon pump itself is achieved via the separate second lumen 166 in the catheter.

For a proper functioning of such a system, it is necessary to have a very fast inflation of the first occlusion balloon 161, while the second balloon 163 just starts to inflate with some time delay. Preferably, the majority of the inflation of the second balloon 163 takes place while the occlusion is complete. This ensures a correct unidirectional flow into the lower descending aorta.

Upon deflation, the deflation of the first occlusion balloon 161 has to be more rapid than for the second balloon 163 as well, thus creating a lower pressure in the left ventricle when the decreasing volume of the large balloon gives an unloading effect on this ventricle.

Instead of using two separate drivers for both balloons 161, 163, the solution to create a right timing of inflation and deflation is found in choosing the right dimension ratio of the cross section of lumens 165 and 166. This permits the device 160 to work on a standard single driver unit, like the ones manufactured by Arrow International Inc., Reading, Pa. or Data scope Corporation, Mahwah, N.J., which are widespread in hospitals all over the world.

The proposed solution in the present disclosure is that the capacity of the single driver is sufficient to give full pressure to both lumens 165 and 166 and that they inflate and deflate completely independently of each other. When the volume of the first occlusion balloon 161 is only 25% of the volume of the second balloon 163, while the cross-section area of both lumens 165 and 166 is identical, the timing sequence is regulated automatically by this dimension ratio.

A typical example of the embodiment described above is the use of a 10 cc occlusion balloon with outer diameter 30 mm in combination with a 40 cc balloon with an outer diameter of 15.5 mm. Both inflation lumens have equal cross section area and the system is directly connected to a driver that is adjusted for a 50 cc balloon capacity.

The first occlusion balloon 161 will inflate rapidly to its maximum pressure and full occlusion, giving counter pulsation to the coronary arteries, while the inflation of the second balloon 163 follows at a relatively slower rate. Then the second balloon 163 will push the blood only into the lower aorta.

Upon deflation, the same speed and timing difference ensures that first the occlusion stops and the underpressure of the deflating first occlusion balloon 161, followed by the deflation of the second balloon 163 unloads the left ventricle, exactly as is desirable. Off course other ratio's than the 1 to 4 volume difference between the small and large balloons 161, 163 and also the ratio between the cross-section area of lumens 165 and 166 can simply be modified to achieve different inflation and deflation sequences. For example, the ratio of the balloon volume may range somewhere between approximately 1:3 until 1:5. The ratio of the cross-section area of lumen 165 compared to lumen 166 may range between approximately 0.75 until 1.5. However, it is very simple to vary these sizes and all devices will be suitable for use on a single standard driver unit.

In another embodiment, a different solution can be achieved by using only one inflation lumen for both balloons 161, 163, with a flow restriction between the balloons 161, 163 that causes a delay in the timing of the inflation and deflation process of the second balloon 163 as compared to the first occlusion balloon 161.

In FIG. 3 a cross section of the aorta 150 plus such an alternative device 170 is schematically shown. The catheter has a cross section B-B showed in FIG. 4, with one small lumen 171 for a guidewire and a larger lumen 172 for inflation and deflation, preferably with helium as medium.

One or more inflation holes 173 are directly ending inside the relatively short first occlusion balloon 174. The relatively long second balloon 175 does not have inflation holes that connect directly to the catheter lumen 172. Instead, between the two balloons 174 and 175 a small restriction 176 is built in, with one or more small holes 177 that allow the medium to flow from the inside of the first occlusion balloon 174 into the inside of the second balloon. The size and number of these restriction holes 177 can be chosen in such a way that the inflation of the second balloon 175 is delayed when the first occlusion balloon 174 is inflated.

In the other part of the cycle the deflation of the first occlusion balloon 174 goes rapidly, followed by a slower deflation of the second balloon 175. Fine tuning of the restriction holes 177 give an improved pumping effect with the first occlusion balloon 174 acting as a kind of remotely controlled active stop valve. The entire volume change of the second balloon 175 upon its inflation will be directed downstream as long as the first occlusion balloon 174 completely occludes the artery.

The restriction opening 176 may have holes 177 with a specific fixed size, but it can also be made adjustable, like in a needle valve or by clamping it with an additional outer collar 178 that elastically changes the size of holes 177. When the sequence of the inflation and deflation is chosen well compared to the heartbeat sequence, the first occlusion balloon 174 fully occludes during diastole, followed by the inflation of the second balloon 175. Therefor the total antegrade blood flow will increase. During a part of systole both balloons can be empty and the blood coming from the heart can almost freely flow around the empty balloons. At the very end of the systole the first occlusion balloon 174 can then be inflated, causing some afterload that improves the perfusion in the coronary and carotid arteries at diastole. By this configuration, the second balloon 175 need only be inflated as soon as the pressure in the first occlusion balloon 174 has become high enough to create a flow through the restriction holes 177. Therefore, the sizing of the components automatically generates the desired timing sequence and the first occlusion balloon 174 will reach full occlusion before the second balloon 175 becomes inflated.

The total flow depends on the sizes of the balloons 174, 175, the dimensions of the catheter lumens, the inflation holes and the driver settings for the pumping frequency. Another embodiment of the disclosure is that the better flow output with occluder balloons enables the downsizing of the present balloon, which is an advantage. The flow direction can also be influenced by the way of insertion of the system, either through the subclavian or femoral artery. In case of insertion through the subclavian artery, the relative position of the second and first occlusion balloons 175, 174 on the catheter has to be switched, again with the first occlusion balloon 174 positioned closest to the aortic root.

Other combinations of balloons and valves can be made as well. For example, a long IABP balloon combined with two small occlusion balloons on the same catheter may cause a better, more stable positioning of the system in the aorta. The sizes of such balloons may be different from each other, dependent on the body location, where they are used. Eventually additional expandable Nitinol frames with or without valves can be used to keep the balloons centered in the lumen in order to avoid energy loss and whipping of the balloon against the inner wall and/or cause a more stable anchoring to avoid longitudinal movements.

While using balloon pumps according to the disclosure it is not always necessary to have full occlusion. In many cases, it is better to still have some leakage through or around the small balloon, while it still creates a sufficient counter-pulsation for the blood supply into the coronary arteries. This may be used if it is not desirable to create much radial pressure to the aorta wall.

The examples given in FIGS. 1 through 4 are only some embodiments of an IABP combined with an occlusion balloon. It may be clear that different configurations of such combinations are also meant to be included in the principle of the disclosure.

It is noted that terms like “preferably”, “generally” and “typically” are not utilized herein to limit the scope of the claims or to imply that certain features are critical, essential, or even important to the structure or function of the claims. Rather, these terms are merely intended to highlight alternative or additional features that may or may not be utilized in a particular embodiment of the present disclosure. Likewise, for the purposes of describing and defining the present disclosure, it is noted that the terms “substantially” and “approximately” and their variants are utilized herein to represent the inherent degree of uncertainty that may be attributed to any quantitative comparison, value, measurement or other representation, as well as to represent the degree by which a quantitative representation may vary without resulting in a change in the basic function of the subject matter at issue.

While certain representative embodiments and details have been shown for purposes of illustrating the disclosure, it will be apparent to those skilled in the art that various changes may be made without departing from the scope of the disclosure, which is defined in the appended claims.

1. An intra-aortic balloon pumping device comprising:
a catheter adapted to be inserted into an aorta and having a separated first and second lumen therein adapted for passage of a driving gas;
a short small volume first occlusion balloon with a relatively large outer diameter, sufficient to occlude the aorta when inflated, positioned on a distal portion of the catheter and having at least one aperture formed in the catheter for communication of driving gas between the first lumen and a chamber defined by the first occlusion balloon;
a long larger volume second balloon with a smaller outer diameter than the aorta when inflated, positioned on a more proximal portion on the catheter and having at least one aperture formed in the catheter for communication of driving gas between the second lumen and a chamber defined by the second balloon; and
a driver unit that is coupled to the first and second lumen at a proximal portion of the catheter for pumping the driving gas into and out from the first and second lumen to inflate and deflate the first and second balloons such that the balloons inflate in sequence from distal to proximal and then deflate in sequence from distal to proximal, wherein the cross section area of the first lumen is substantially equal to the cross section area of the second lumen.

2. The device of claim 1 wherein the apertures associated with the first and second balloon chambers are sufficient large to enable a substantially unobstructed flow of the driving gas through the apertures.

3. The device of claim 1, wherein the ratio of the maximum volumes of e first and second balloon ranges from 1:3 until 1:5.

4. The device of claim 3, wherein the maximum volume of the first occlusion balloon ranges from 5 to 15 cc.

5. The device of claim 1, wherein the ratio of the cross-section area of the first and second lumen ranges from 0.75 until 1.5.

6. The device of claim 1, wherein the first occlusion balloon is positioned proximal of the second balloon.

7. The device of claim 1, wherein the first occlusion balloon is replaced by an inflatable valve.

8. An intra-aortic balloon pumping device comprising:
a catheter adapted to be inserted into an aorta and having a single lumen therein adapted for passage of a driving gas;
a short small volume first occlusion balloon with a relatively large outer diameter, sufficient to occlude the aorta when inflated, positioned on a distal portion of the catheter and having at least one aperture formed in the catheter for communication of driving gas between the lumen and a chamber defined by the first occlusion balloon;
a long larger volume second balloon with a smaller outer diameter than the aorta when inflated, positioned on a more proximal portion on the catheter and having only at least one aperture formed in the catheter for communication of driving gas between the chamber defined by the first occlusion balloon and the chamber defined by the second balloon; and
a driver unit that is coupled to the lumen at a proximal portion of the catheter for pumping the driving gas into and out from the lumen to inflate and deflate the first and second balloons such that the balloons inflate in sequence from distal to proximal and then deflate in sequence from distal to proximal; and wherein the cross section area of the first lumen is substantially equal to the cross section area of the second lumen, wherein the second balloon chamber is not directly connected to the lumen, other than via the chamber defined by the first occlusion balloon.

9. The device of claim 8, wherein the size of the at least one aperture between the first occlusion balloon and the lumen is sufficient large to enable a substantially unobstructed flow of the driving gas through the apertures.

10. The device of claim 8, wherein the size of the at least one aperture between the first occlusion balloon and second balloon is adjustable in order to regulate the timing of inflation and deflation.

11. The device of claim 8, wherein the size of the at least one aperture between the first occlusion balloon and second balloon is sufficient small to cause a fast and strong inflation effect on the first occlusion balloon while the second balloon is inflating more slowly.

12. The device of claim 11, wherein the size of the at least one aperture between the first occlusion balloon and second balloon is sufficient small to cause a fast deflation effect on the first occlusion balloon while the second balloon is deflating more slowly.

13. The device of claim 8. wherein the first occlusion balloon is replaced by an inflatable valve.
